(19) United States
(12) Patent Application Publication
(10) Pub. No.: US 2016/0198973 A1
(43) Pub. Date: Jul. 14, 2016

(54) Electrophysiological Assessment of Learning

(71) Applicant: Vanderbilt University, Nashville, TN (US)

(72) Inventors: Keisuke Fukuda, Nashville, TN (US); Geoffrey F. Woodman, Nashville, TN (US)

(21) Appl. No.: 14/993,356

(22) Filed: Jan. 12, 2016

Related U.S. Application Data
(60) Provisional application No. 62/102,716, filed on Jan. 13, 2015.

(51) Int. Cl. A61B 5/0484; A61B 5/0478

(52) U.S. Cl. CPC A61B 5/04842; A61B 5/0478

(57) ABSTRACT

Methods and systems therefrom for improved learning via electrophysiological assessment of learning are provided. The method includes generating, during a first time period, at least one sensory stimulus for a learner, collecting, during a second time period after the first time period, first electroencephalogram (EEG) signals for at least one first electrode site and second EEG signals for at least one second electrode site, calculating a first characterization value based on the first EEG signals and a second characterization value based on the second EEG signals, determining whether the first characterization value and the second characterization value fail to meet respective first and second conditions, and, in response to determining that the first characterization value and the second characterization value fail to meet the respective first and second conditions, regenerating the at least one sensory stimulus for the user.

### CROSS-REFERENCE TO RELATED APPLICATIONS

This application claims priority to and the benefit of U.S. Provisional Patent Application No. 62/102,716, filed Jan. 13, 2015 and entitled “Online Electrophysiological Assessment of Learning,” the contents of which are hereby incorporated by reference in their entirety as if fully set forth herein.

### GOVERNMENT LICENSE RIGHTS

This invention was made with government support under contract/grant/award numbers R01-EY019882, awarded by the National Eye Institute, and BCS 09-57072, awarded by the National Science Foundation. The government has certain rights in the invention.”

### FIELD OF THE INVENTION

The present invention relates to, and more specifically to apparatus and methods for assessment of learning and more particularly, to apparatus and methods for improving learning via electrophysiological assessment of learning.

### BACKGROUND

Cognitive neuroscientists have found several encoding-related neural signals that differentiate remembered items from items that are later forgotten. Specifically, recordings of the electroencephalogram (EEG) and averaged event-related potentials (ERPs) have provided two excellent candidates. First, a larger sustained positivity has been observed at frontal electrodes during encoding for items that are later remembered than for items that are later missed. Second, it has also been observed that alpha-band activity is more suppressed during encoding for items that are later remembered than for those that are later missed. However, Even though these two neural measures of the quality of memory encoding are well established, there has been no conclusive evidence that either measure can be utilized in real time to predict whether a stimulus will be remembered. Further, it has remained unknown whether electrophysiological memory effects are of sufficient magnitude to predict subsequent memory after a single visual stimulus is encoded.

### SUMMARY

The present technology is directed to system and methods for monitoring the fluctuations of the electroencephalogram (EEG) during encoding, and forecasting, on a single-trial basis, the likelihood that a given item will be later recognized. Moreover, present technology is also direct to systems and methods for identifying items that are poorly encoded in a learners memory (i.e., forecasted to be forgotten) by electro physiological measures, to enhance recognition memory by having learners restudy the poorly encoded items.

In a first embodiment, there is provided a computer-implemented method. The method includes generating, during a first time period, at least one sensory stimulus for a learner and collecting, during a second time period after the first time period, first electroencephalogram (EEG) signals for at least one first electrode site and second EEG signals for at least one second electrode site. The method further includes calculating a first characterization value based on the first EEG signals and a second characterization value based on the second EEG signals. The method further includes determining whether the first characterization value and the second characterization value fail to meet respective first and second conditions and, in response to determining that the first characterization value and the second characterization value fail to meet the respective first and second conditions, regenerating the at least one sensory stimulus for the user.

In some implementations of the first embodiment, the first EEG signals can be collected from an electrode over the frontal region of the brain of the learner and the first characterization value can be based on event-related potential (ERP) signals derived from the first EEG signals. Further, the second EEG signals can be collected from an electrode over the occipital region of the brain of the learner and the second characterization value can be based on occipital alpha power signals derived from the second EEG signals.

In some implementations of the first embodiment, the at least one sensory stimulus can include a visual sensory stimulus.

In some implementations of the first embodiment, the calculating can include selecting the first characterization value to be a first Z-score based on the first EEG signals, and selecting the second characterization value to be a second Z-score based on the second EEG values. Further, the first condition can be that the first Z-score be less than or equal to a first Z-score threshold value and the second condition can be that the second Z-score be greater than or equal to a second Z-score threshold value.

In a second embodiment, there is provided a system including at least one user interface device, an electroencephalogram (EEG) signal interface, a processor communicatively coupled to the at least one user interface and the EEG signal interface; and a computer-readable medium, having stored thereon a plurality of instructions for causing the processor to perform the steps of the method of the first embodiment.

In a third embodiment, there is provided a non-transitory computer readable medium, having stored thereon a computer program executable by a computing device, the computer program comprising a plurality of code sections for causing the computing device to perform the method of the first embodiment.

### BRIEF DESCRIPTION OF THE DRAWINGS

FIG. 1 shows an EEG-enhanced learning system in according to the present technology;

FIG. 2 is a flowchart of steps in an exemplary method according to the present technology;

FIGS. 3A and 3B show a sample trial sequence that is useful for understanding the present technology;

FIGS. 4A and 4B show the electroencephalogram results that are useful for understanding the present technology;

FIGS. 5A, 5B, 5C, 5D, 5E, and 5F show frontal positivity and results that are useful for understanding the present technology;

FIGS. 6A and 6B heat maps depicting the combined predictive power of the frontal positivity and the occipital alpha power;

FIGS. 7A, 7B, 7C, and 7D show the electroencephalogram results that are useful for understanding the present technology;

FIGS. 8A, 8B, and 8C show performance from a recognition test; and

FIG. 9A and FIG. 9B illustrate exemplary possible system configurations.

### DETAILED DESCRIPTION

The present technology is described with reference to the attached figures, wherein like reference numerals are used throughout the figures to designate similar or equivalent elements. The figures are not drawn to scale and they are provided merely to illustrate the various aspects the present technology. Several aspects of the present technology are described below with reference to example applications for illustration. It should be understood that numerous specific details, relationships, and methods are set forth to provide a full understanding of the present disclosure. One having ordinary skill in the relevant art, however, will readily recognize that the present technology can be practiced without one or more of the specific details or with other methods. In other instances, well-known structures or operations are not shown in detail to avoid obscuring the present technology. The present technology is not limited by the illustrated ordering of acts or events, as some acts may occur in different orders and/or concurrently with other acts or events. Furthermore, not all illustrated acts or events are required to implement a methodology in accordance with the present technology.

As discussed above, the utility of various types of EEG signals for forecasting encoding has been unsuccessfully studied. However, the inventors have discovered that when one simultaneously measures a particular set of different electrophysiological signals, these signals provide a reliable and dissociable utility for predicting subsequent memory encoding. In particular, by combining EEG signals associated with memory encoding and EEG signals associated with processing of sensory stimuli. Thus, the various embodiments are directed to systems and methods for improving learning based in the foregoing. In particular, using at least two such EEG signals to identify items that need restudying during a learning episode and thereafter intervening in the learning process in order to improve recognition memory. For example, by identifying and re-presenting items requiring restudy to the learner.

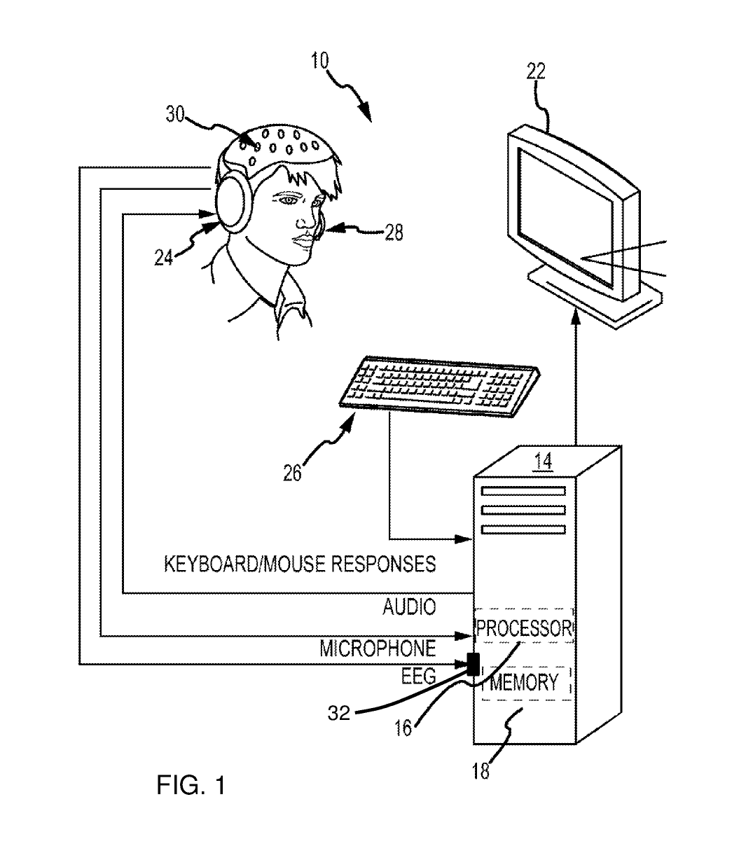

Referring now to FIG. 1, an embodiment of an EEG-enhanced learning system 10 comprises the hardware and software modules of the learning system plus additional hardware and software modules to implement the EEG-based enhancement. Although learning system, 10 will be illustrated with respect to the components shown in FIG. 1, the present technology contemplates that a learning system in accordingly with other embodiments can include more or less components than illustrated in FIG. 1. Moreover, it should be noted that the components shown in FIG. 1 are presented separately for ease of illustration. Thus, two or more of the components illustrated in FIG. 1 can be embodied in a single component.

The learning system 10 itself comprises one or more computers 14 including one or more processors 16 and memory 18. A learning module 20 including information for generating sensory stimuli and an EEG analysis module 34 for analyzing EEG signals each reside in memory 18 and are executable by processors 16. In operation, the learning module 20 is executed by the processors to generate a representation of the stimuli via at least one of a display 22 and headphones 24 (or audio speakers).

Although FIG. 1 shows a local computer 14, the present disclosure contemplates that the computer 14 can be implemented using any number of remote and/or local computing device communicating over a communications network.

In the exemplary learning system 10, the sensory stimuli can include one or more audio stimuli provided via headphone 24, one or more visual stimuli provided via display 22, or any combinations thereof. However, in other learning systems in accordance with the present technology, the learning module 20 (and the learning system 10) can be configured to generate, alternatively or additionally, other types of sensory stimuli. For example, such stimuli can also include olfactory, tactile, thermoreceptive, nociceptive (i.e., pain), and equilibrioceptive (e.g., balance) stimuli, to name a few.

In response to the presentation of the stimuli to the learner, to test recollection and/or recognition, the learning module can acquire data from the learner via a keyboard/mouse 26 or a microphone 28. However, the present disclosure contemplates that any other type of user interface devices can be used with the present technology. The learning module 20 evaluates the learner's responses to assess the learner's strengths and weaknesses regarding recollection and/or recognition of various types of stimuli. Further, the learning module 20 can interact with EEG analysis module 34, as described below in greater detail to assess whether the learner requires a stimuli to be re-presented to allow restudy and ensure learning thereof.

In some configurations, feedback from the user is not needed. That is, the learning module 20 and EEG analysis module 34 can be configured to run based on the brain data alone to determine whether the learner will remember a specific stimulus. That is, the types of signal contemplated for use in the various embodiments provide a sufficient difference between learned and unlearned stimulus such that the particular EEG patterns for such signals can be evaluated to discern when the learner will remember a specific stimulus.

As shown in FIG. 1, the EEG-based enhancement system can include a cap of EEG electrodes 30 placed on the learner's scalp to continuously provide EEG signals associated with two or more areas of the learner's brain. Moreover, any other type of apparatus for acquiring the necessary EEG signals can be used with the present technology. However, the minimal number of active electrodes needed for the system is two, with one reference electrode. More than be used, but this could be done with a few and three electrodes total (one frontal, one occipital, and a reference place at any location of convenience).

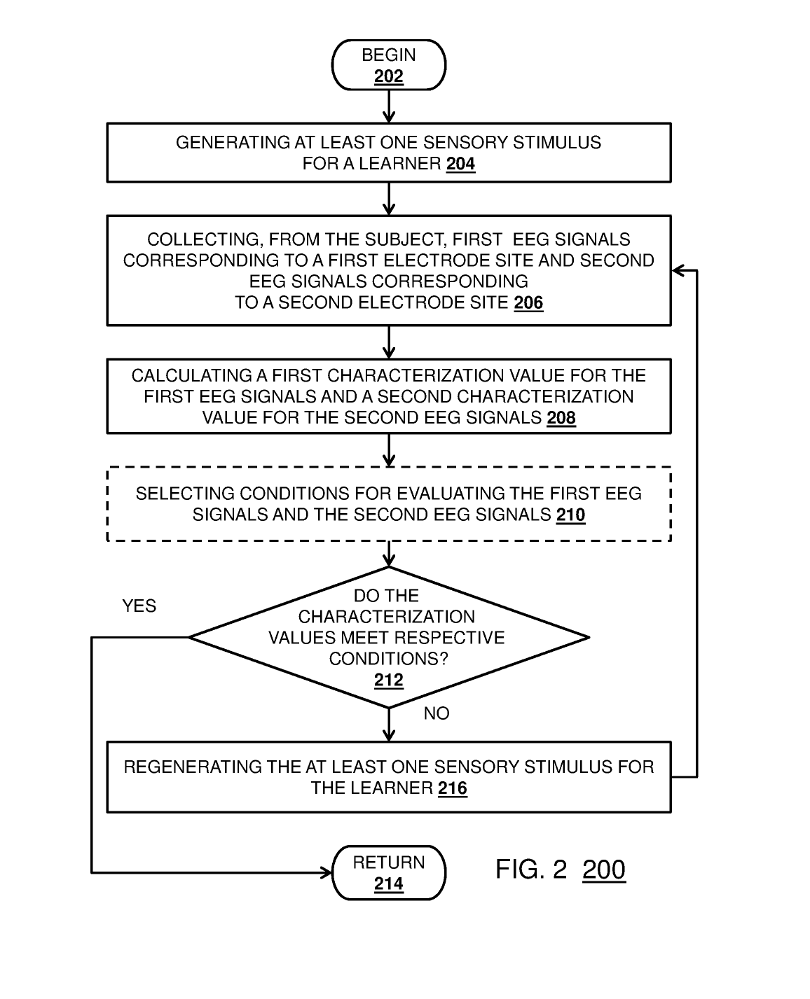

The EEG signals are provided to computer 14 via an EEG signal interface 32. The EEG analysis module 34 is configured to receive the EEG signals from the EEG signal interface 32 and determine whether or not the EEG signals indicate that a learner's exposure to a stimulus is sufficient to ensure future recollection and/or recognition of the stimulus. In this manner, the EEG analysis module 34 can operate cooperatively with the learning module 20. For example, the determinations made by the EEG analysis module 34 can be communicated to the learning module 20. Based on the determination received from the EEG analysis module 34, the learning module can then be configured to re-present the stimulus to improve likelihood by the learner of future recollection and/or recognition of the stimulus. The operation of the learning module 20 and the EEG analysis module 34 is illustrated below in greater detail with respect to FIG. 2.

FIG. 2 is a flow chart of steps in an exemplary method 200 for EEG-enhanced learning in accordance with the present technology. The various steps of method 200 can be incorporated into learning module 20, EEG analysis module 34, or another module incorporating the function of modules 20 and 34. The method 200 can begin at step 202 and proceed to step 204. At step 204, at least one sensory stimulus is generated for a learner. That is, the information to be learned is provided to the learner. For example, as discussed above, step 204 can involve the computer 14 generating, via display 22, visual information for the learner to view, such as words, images, videos, or any combination thereof. Additionally or alternatively, step 204 can involve the computer 14 generating, via headphones 24 (or other audio output device), audio information for the learner to listen to.

After the sensory stimulus is presented at step 204, the method 200 can proceed to step 206. At step 206, EEG signals can be collected from the learning via EEG electrodes 30 from multiple electrode sites associated with the learner's brain. In particular, at least one site can correspond to a site associated with a frontal lobe of the brain of the learner and at least one other site can correspond to a site associated with the occipital lobe of the brain of the learner.

In one particular embodiment, signals can be collected from the Fz site (working memory site) and the O2 site (visual processing site), as defined under the International 10-20 system for electrode placement on a human scalp, when visual information is being presented to the user. The signals from the Fz site can be used to obtain the event-related potential (ERP) for frontal positivity as a function of time after the stimulus in step 204. The signals from the O2 site can be used to obtain the occipital alpha power as a function of time after the stimulus in step 204. However, the present disclosure contemplates that the necessary signals representing working memory and sensory processing can be collected via other locations. For example, any other F or O sites can be used in certain embodiments.

Once the EEG signals are collected at step 206, the EEG signals can be characterized at step 208 by calculation of corresponding characterization values. In particular embodiments, the characterization values can be Z-scores, percentile values, or raw amplitudes derived from each of the EEG signals. All quantifications work, although Z-scores are more reliable when the amount of data is limited, such as early in a learning session. In particular, Z-scores for the measurements of ERP and occipital alpha power obtained from the EEG signals. However, the present disclosure contemplates that other types of values can be calculated for characterizing the EEG signals.

Once the characterization values for the EEG signals are obtained at step 208, a determination of whether such characterization values meet respective conditions is made at step 212. In particular, conditions indicating that there is a high likelihood or confidence of recollection or recognition of the stimulus by the learner.

Optionally, at step 210, the conditions can be selected. For example, threshold conditions can be specified that indicate a value required by the characterization values of step 208 to indicate that there is a high likelihood or confidence that recollection or recognition of the stimulus will occur, i.e., that the learner has learned. For example, a minimum or maximum values derived from the EEG signals. For example, in a particular configuration, the conditions can specify that the values associated with frontal positivity indicate being in the bottom 40% percentile and that the values associated with occipital alpha power indicate being in the top 40% percentile.

In some embodiments, the conditions can be predefined. In other embodiments, the conditions can be computed based on input parameters. For example, input parameters for computing a threshold can be provided. As conditions may vary from learner to learner, learner-specific input parameters may be provided in some embodiments. In some cases, such learner-specific input parameters may be known a priori. In other cases, the input parameters can be identified during a learning session. That is, as the learner's responses to stimuli are monitored and studied over time, the input parameters may be adjusted to account for the matter in which the learner's EEG responds to stimuli.

For example, over time, the classification of each set of EEG signals as indicating the need for restudy or no restudy can change over time. Thus, as more EEG signals are collected, the estimation of underlying distribution of each signal becomes more precise. This allows for a better calibration of the threshold value for each signal should be. For example, some items that were classified as not needing restudy might become classified as needing restudy as threshold values become more precise.

Referring back to step 212, if the characterization values meet their respective conditions the method 200 can proceed to step 214 and resume previous processing, including repeating method 200 for other stimuli. If the characteristic values fail to meet their respective conditions the method 200 can instead proceed to step 216. At step 216, the stimulus is regenerated, i.e. re-presented to the learner for restudy. The method 200 can then proceed back to step 206 to repeat such re-representing of the stimulus until the conditions at step 212 are met.

In some embodiments, rather than proceeding from step 212 to 216, method 200 may be first repeated one or more times with different stimuli. Thereafter, the method may then be repeated for any stimuli requiring restudy at a later time.

The method 200 can be embedded in a computing device (e.g., computer 14) as part of an algorithm. For example, an exemplary algorithm for determining whether or not a stimulus needs to be re-presented or restudied is as follows (assuming EEG signals from Fz and O2 sites and Z-scores as discussed above).

Step 1—Calculate the criteria value (in Z-score) by computing:

z_fp_criteria = norminv(criteria_frontal_positivity,0,1);
z_oa_criteria = norminv(1−criteria_occipital_alpha_power,0,1);
cdf_fp_criteria = normcdf(z_fp_criteria);
cdf_oa_criteria = normcdf(z_oa_criteria);

Step 2—Calculate characterization values for the EEG signals

```
z_current_fp =
  normcdf(current_frontal_positivity,mean_frontal_positivity,
  stdev_frontal_positivity);
z_current_oa =
  normcdf(current_occipital_alpha_power,mean_occipital_alpha_power,
  stdev_occipital_alpha_power);
```

Step 3—Determine whether characterization values indicate re-representation or restudy of the stimulus is needed.

```
if z_current_fp <= cdf_fp_criteria && z_current_oa >=
cdf_oa_criteria
    restudy_or_not = 1;  %restudy!
else
    restudy_or_not = 0;  %no restudy!
end;
```

Where
restudy_or_not=1 indicates that the stimulus needs to be restudied;
restudy_or_not=1 indicates that the stimulus does not need to be restudied;
X=norminv(P,mu,sigma) is a function that computes the inverse of the normal cumulative distribution function (cdf) using the corresponding mean mu and standard deviation sigma at the corresponding probabilities in P; and
p=normcdf(x,mu,sigma) is a function that returns the normal cdf at each value in x using the specified values for the mean mu and standard deviation sigma.
   and where
current_frontal_positivity (frontal positivity (Fz ERP) for a current trial): The voltage difference between a frontal electrode and the reference measured during the first several seconds after the stimulus appears;
mean_frontal_positivity (mean value of frontal positivity recorded): The average voltage difference between a frontal electrode and the reference measured during the first several seconds across all of the stimuli shown;
stdev_frontal_positivity (standard deviation of frontal positivity recorded): The variation of the voltage difference between a frontal electrode and the reference measured during the first several seconds across all of the stimuli shown;
current_occipital_alpha_power (occipital alpha power (O2 alpha power) for a current trial): The power measured between 8-12 Hz between an occipital electrode and the reference during the first several seconds after the stimulus appears;
mean_occipital_alpha_power (mean value of occipital alpha power recorded): The average power measured between 8-12 Hz between an occipital electrode and the reference during the first several seconds across all of the stimuli shown;
stdev_occipital_alpha_power (standard deviation of occipital alpha power recorded): The variation of the power measured between 8-12 Hz between an occipital electrode and the reference during the first several seconds across all of the stimuli shown;
criteria_frontal_positivity (input parameter for computing threshold values for frontal positivity): The user can select how well they want the stimuli to be learned, for example, such that only the worst learned 25% of stimuli are relearned, or that the worst learned 50% of stimuli are relearned, etc. In certain embodiments, this should be set as a percentile value divided by 100 from the lowest. (e.g., 0.4=40th percentile from the lowest value); and
criteria_occipital_alpha_power (input parameter for computing threshold values for occipital alpha power): In certain embodiments, this should be set as a percentile value divided by 100 from the highest value. (e.g., 0.4=40th percentile from the highest value).

### EXAMPLES

The examples shown here are not intended to limit the various embodiments. Rather they are presented solely for illustrative purposes.

Experiment 1

In Experiment 1, a determination was made as to whether the two electro-physiological measures index the same or separable mechanisms operating at encoding. Experiment 1 also served the broader goal of establishing the feasibility of using these measures to forecast the later recognition of a particular stimulus, the question addressed directly in Experiment 2.

Method

Stimuli and Procedures.

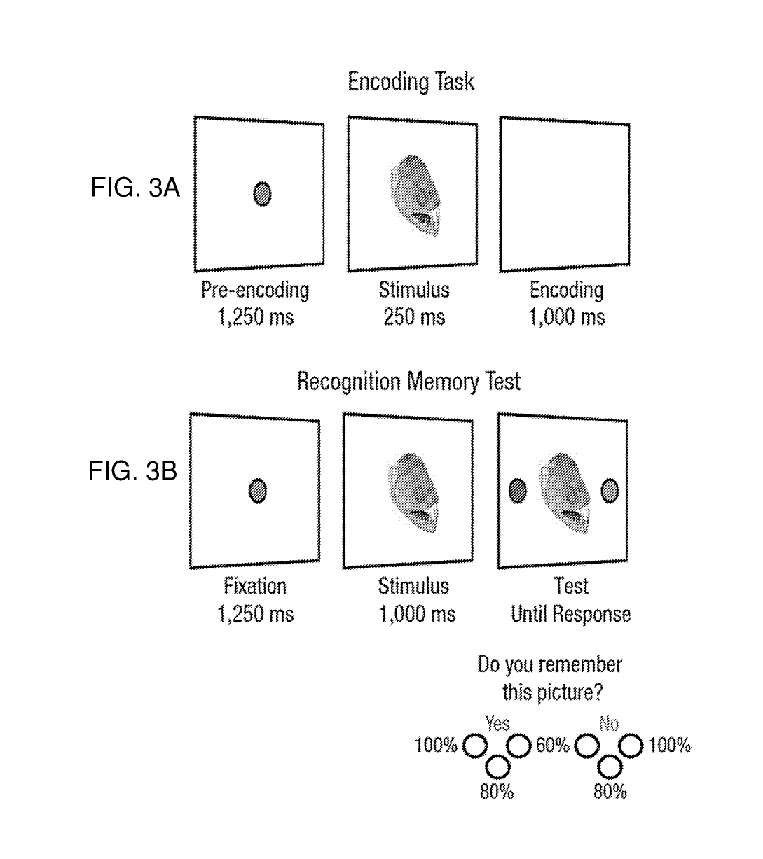

The stimuli and tasks are illustrated in FIGS. 3A and 3B, which show a sample trial sequence from the encoding task (FIG. 3A) and the recognition memory test (FIG. 3B) in Experiment 1. In the encoding task, a fixation point (left in FIG. 3A) was followed by a picture of a real-world object (middle in FIG. 3A) and then a blank interval (right in FIG. 3A) for encoding. After completing all encoding trials, participants performed the recognition memory test, in which they used buttons on a game pad to indicate with 100%, 80%, or 60% confidence whether or not they had seen a picture during encoding. The position of the red and blue dots in FIG. 3B (to the left and right of the stimulus, respectively, at right in FIG. 3B) indicated which side of the game pad to use in making their response.

The stimuli were adapted from a published set of photographs. During the encoding task, participants were sequentially presented with 500 pictures of real-world objects with short breaks every 50 pictures. They were instructed to study each item while holding central fixation so that they could later perform a recognition memory test. Participants initiated each trial by pressing a button on a game pad. After a 1,250-ms pre-encoding period, in which the screen was blank except for a central fixation dot, a picture was presented for 250 ms. The picture was followed by a 1,000-ms encoding period, during which the computer screen remained blank. After the encoding task, participants' resting-state EEG activity was measured when their eyes were open and closed for 15 min. Then, participants' memory for the pictures was tested.

The recognition memory test started with the onset of a central fixation dot (left in FIG. 3B). Participants initiated each test trial by pressing a button on the game pad. They were instructed to maintain central fixation without blinking until each trial was over. Following a 1,250-ms blank period, a picture of a real-world object was presented at the center of the screen (middle in FIG. 3B, new and old pictures were randomly interleaved across trials). After 1,250 ms, a blue and a red dot appeared, one on each side of the picture (right in FIG. 3B). Participants indicated whether they remembered seeing this picture during the study phase by pressing one of three buttons on the side of the game pad indicated by the position of the dot. The red dot indicated which buttons to press if they remembered seeing the picture, and the blue dot indicated which buttons to press if they did not. Of the three buttons on each side, the outermost indicated 100% confidence in their judgment, the middle button indicated 80% confidence, and the inner button indicated 60% confidence (see FIG. 3B). The sides on which the red and blue dots appeared were randomized from trial to trial. After the response, the trial was over, and participants were provided with a self-determined interval to rest their eyes and blink. Participants were tested on 500 studied pictures and 250 new pictures.

Data Acquisition and Analysis.

EEG data were recorded using a right-mastoid reference and were re-referenced off-line to the average of the left and right mastoids. The international 10-20 electrode sites (Fz, Cz, Pz, F3, F4, C3, C4, P3, P4, PO3, PO4, O1, O2, T3, T4, T5, and T6) and a pair of custom sites, OL (halfway between O1 and OL) and OR (halfway between O2 and OR) were used. Eye movements were monitored using electrodes placed 1-cm lateral to the external canthi for horizontal movement and an electrode placed beneath the right eye for blinks and vertical eye movements. The signals were amplified with a gain of 20,000, band-pass filtered from 0.01 to 100 Hz, and digitized at 250 Hz. Trials accompanied by horizontal eye movements (>30 μV mean threshold across observers) or eye blinks (>75 μV mean threshold across observers) were rejected before further analyses.

To measure the ERPs preceding memory encoding, waveforms were time-locked to the button-press response that initiated a trial and examined the waveforms recorded from −1,250 ms to 0 ms relative to the onset of the picture.

These EEG epochs were baseline-corrected to the mean EEG amplitude measured −400 to 0 ms before the beginning of the measurement epoch of interest.

To examine EEG activity during memory encoding, waveforms were time-locked to the onset of memory stimuli and examined the EEG recording from 0 to 1,250 ms following the onset of each memory stimulus. These EEG epochs were baseline-corrected to the mean EEG amplitude −400 to 0 ms relative to the stimulus onset. For presentation purposes, we needed to concisely summarize the relationship between our electrophysiological measures and behavior. As a result, the pre-encoding and encoding signals for each epoch were binned and averaged based on recognition performance in the memory test. The EEG activity recorded as the participants viewed the items that were later recognized with 100% confidence were binned as high-confidence hit trials, and those recorded as the participants viewed the items that were later recognized at lower confidence levels (80% and 60%) were binned as low-confidence hit trials. The EEG segments recorded as the participants viewed the items that were later missed were binned as miss trials. These binned averages also allowed us to confirm that our findings replicated previous reports of the traditional mean amplitudes across these types of trials.

To examine the oscillatory responses, frequency content was measured during the same pre-encoding and encoding epochs described above on a trial-by-trial basis. Spectral decomposition with a fixed window size of 400 ms and a window overlap of 380 ms was performed using the spectrogram.m function in MATLAB (The MathWorks, Natick, Mass.) for each single-trial EEG epoch to obtain the time-frequency representation of the signal. Then, the resultant time-frequency representation for each epoch was sorted into the appropriate high confidence, low confidence, or miss bin.

Results

Behavioral Results.

For studied objects, participants recognized 63% of the stimuli with 100% confidence and 14% of the stimuli at 80% or 60% confidence. Participants failed to recognize the remaining 23% of the stimuli. They successfully rejected 76% of new objects that they had not studied during the encoding phase. Table 1 reports the proportions of trials used to derive the receiver-operating-characteristic (ROC) curves in this experiment.

TABLE 1
Results of Experiment 1: Mean Proportion of Responses on the Recognition Memory Test

| | Response | | | | | |
|---|---|---|---|---|---|---|
| Item type | “100% old” | “80% old” | “60% old” | “100% new” | “80% new” | “60% new” |
| Old | .63 (.03) | .08 (.01) | .06 (.01) | .07 (.02) | .08 (.02) | .07 (.01) |
| New | .10 (.02) | .07 (.01) | .07 (.01) | .35 (.05) | .24 (.03) | .17 (.04) |

Note:
Standard errors are given in parentheses.
Proportions indicate participants' confidence in their old/new judgment.

The mean area under the ROC curve (AUC) was 0.82. These results demonstrate that, on average, participants performed the memory task accurately.

Traditional ERP and EEG Analysis.

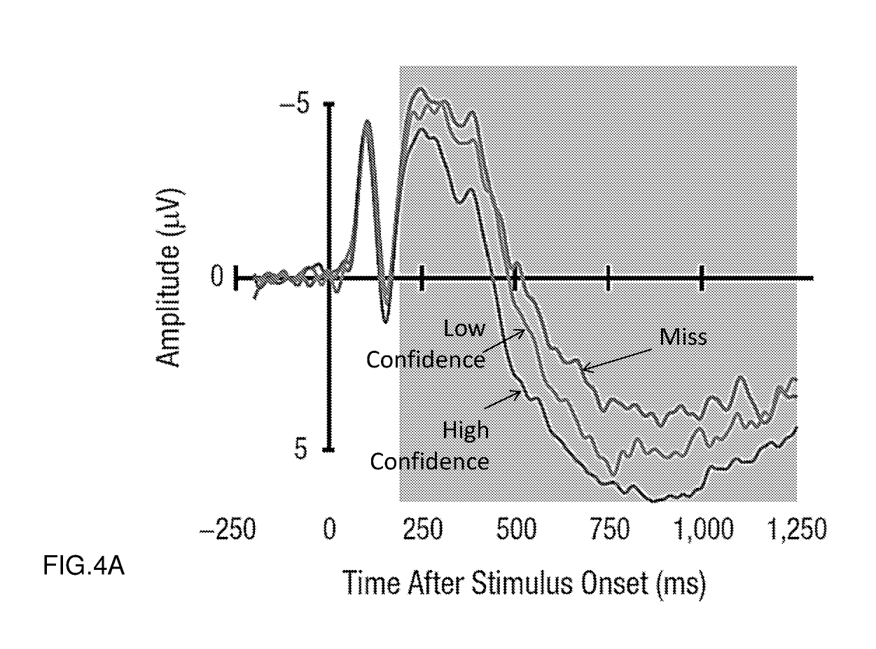

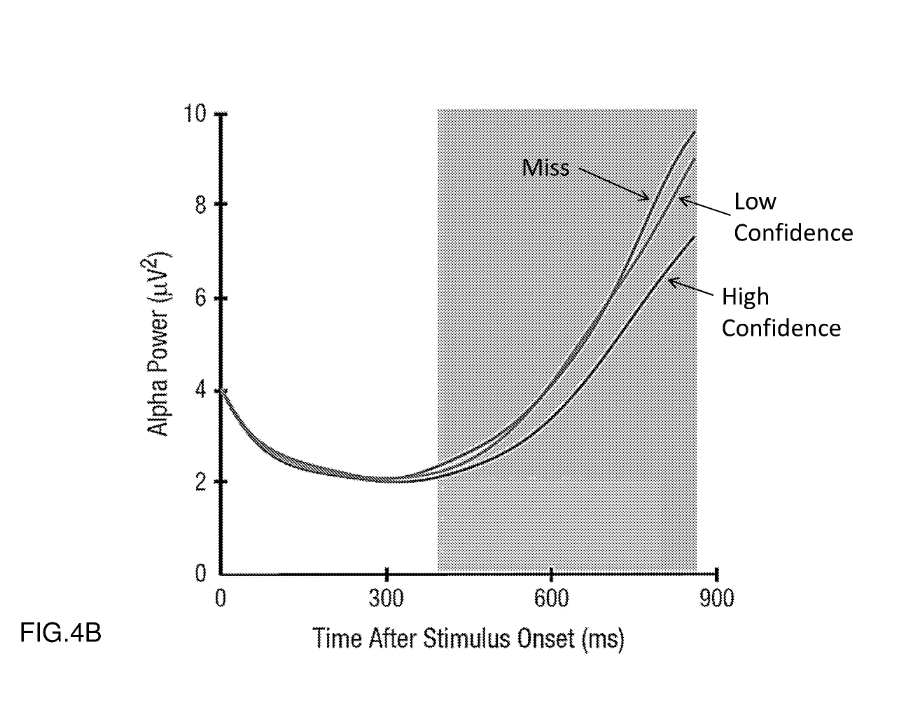

The results of this analysis are illustrated in FIGS. 4A and 4B. FIGS. 4A and 4B show the electroencephalogram results of Experiment 1: mean amplitude at the Fz site (FIG. 4A) and mean alpha power at the O2 site (FIG. 4B) during the encoding task. Gray shading indicates the time window over which data were averaged to quantify the amplitude of each event-related potential. The data points used to plot the curves in FIG. 4B represent the alpha power observed within a 400-ms sliding window that had a 20-ms step size. The values on the x-axis represent the front ends of these time windows.

Using traditional ERP and EEG analyses, it was found that frontal waveforms exhibited a sustained positivity of larger amplitude for high-confidence items than for low-confidence and miss items (see FIG. 4A). The sustained frontal positivity was quantified as the mean amplitude in the time window 200 ms to 1,000 ms after the onset of each studied item at the midfrontal channel (i.e., channel Fz) where the effect was maximal. An analysis of variance (ANOVA) confirmed that this subsequent memory effect was highly significant, F(2, 38)=15.34, p<0.001, ηₚ²=0.45, and was driven by more positive amplitudes in response to the high-confidence items than to both low-confidence items, t(19)=3.30, p<0.01 (95% confidence interval, or CI, for the difference=[0.45, 1.99 μV], Bayes factor=15.5), and miss items, t(19)=5.75, p<0.001 (95% CI for the difference=[1.30, 2.78 μV], Bayes factor=2,349.0). These observations are supported by other work that has examined such differences using conventional mean ERP analyses.

There was a concern that the mean amplitude differences might be driven by the more jittered onset times across participants because of the smaller number of trials for the low-confidence (14% of trials) and miss items (23% of trials) than for the high-confidence items (63% of trials). If this were the case, the amplitude of the frontal positivity measured with the fewest trials (i.e., low-confidence items) should be the lowest because of the largest variability of onset times. However, the fact that the mean amplitude for low-confidence items was significantly higher than the mean amplitude for miss items, t(19)=2.11, p<0.05 (95% CI for the difference=[0.01, 1.63 μV], Bayes factor=1.6) rules out this simple explanation.

Next, the oscillatory activity was examined during the encoding period. As shown in FIG. 4B, the EEG during the encoding period showed a clear suppression of occipital alpha power following the onset of the to-be-remembered items. Occipital alpha power was quantified as the mean power between 8 and 12 Hz in the time window 400 to 1,250 ms after the onset of the study items at a right occipital channel (i.e., channel O2; but this was similar across occipital channels; see the Supplemental Material). An ANOVA confirmed that the occipital alpha power varied as a function of participants' later recognition, F(2, 28)=4.88, p=0.01, ηₚ²=0.20. High-confidence items exhibited lower occipital alpha power than low-confidence items, t(19)=2.12, p<0.05 (95% CI for the difference=[0.01, 1.55 μV2], Bayes factor=1.6) or miss items, t(19)=2.80, p=0.01 (95% CI for the difference=[0.24, 1.69 μV2], Bayes factor=5.7). The only other oscillation that was related to participants' later recognition was a low-frequency frontal effect underlying the aforementioned frontal positivity.

No pre-encoding ERPs or oscillations were predictive of successful memory encoding. This demonstrates that the memory effects were not simply due to tonic changes in brain activity that were present prior to the presentation of the pictures. Instead, these signals reflect the ability of the brain to encode accurate representations of the items immediately following their presentation.

Forecasting Later Recognition of an Object.

The approach in this experiment was to compute measures of successful memory encoding given the magnitude of the frontal positivity and the strength of occipital-alpha-power suppression for each trial. The AUC and the proportion of high-confidence responses to provide diverse measures of successful memory encoding was computed. First, the stimuli were sorted based on the magnitude of each memory-encoding signal. Then, the memory metrics were computed in each quintile bin (i.e., each bin contained 20% of the trials). These measures estimated the strength of encoded memory given the magnitude of the electrophysiological signals.

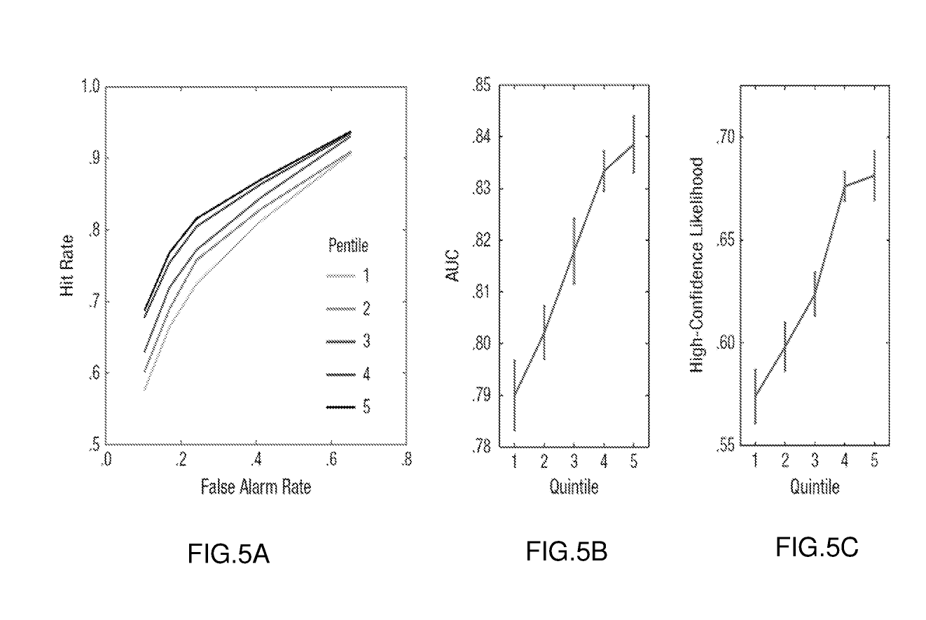

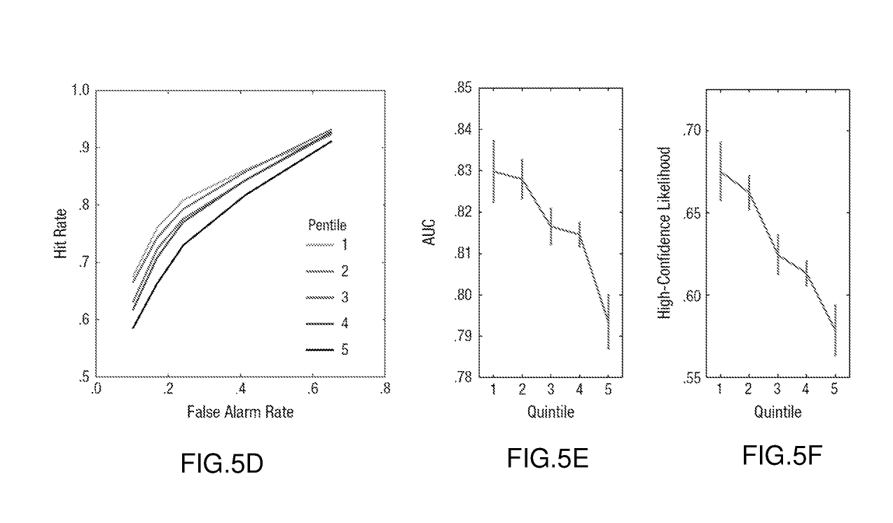

When the trials were sorted by the amplitude of the frontal positivity, there was a monotonic increase in the strength of encoded memory as a function of its magnitude (FIG. 5A). A significant increase was observed in the AUC from the first quintile (M=0.79) to the fifth quintile (M=0.84), F(4, 76)=9.63, p<0.001, ηₚ²=0.34 (FIG. 5B), and the likelihood of a high-confidence response showed a similar increase, from 58% in the first quintile to 68% in the fifth, F(4, 76)=14.15, p<0.001, ηₚ²=0.43 (FIG. 5C). When trials were sorted by the magnitude of the occipital alpha power, there was a highly significant monotonic decline in the memory strength as a function of the alpha power (FIG. 5D). Also observed was a significant decrease in the AUC from the first quintile (M=0.84) to the fifth quintile (M=0.79), F(4, 76)=8.97, p<0.001, ηₚ²=0.32 (FIG. 5E), and the likelihood of a high-confidence response showed a similar decrease, from 68% in the first to 58% in the fifth quintile, F(4, 76)=6.38, p<0.001, ηₚ²=0.26 (FIG. 5F). These results demonstrate the reliability of both the frontal positivity and the occipital alpha power as predictors of subsequent recognition memory when measured on each trial.

To test for independence between the frontal positivity and the occipital alpha power, the correlation between the two signals across trials within each participant was examined. Although the correlation coefficient was reliably different from zero (M=−0.06), t(19)=−4.33, p<0.001 (95% CI=[−0.08, −0.03], Bayes factor=132.1), the relationship accounted for less than 0.3% of the variance. This negligible correlation between the two electrophysiological signals suggests that they index dissociable aspects of memory encoding.

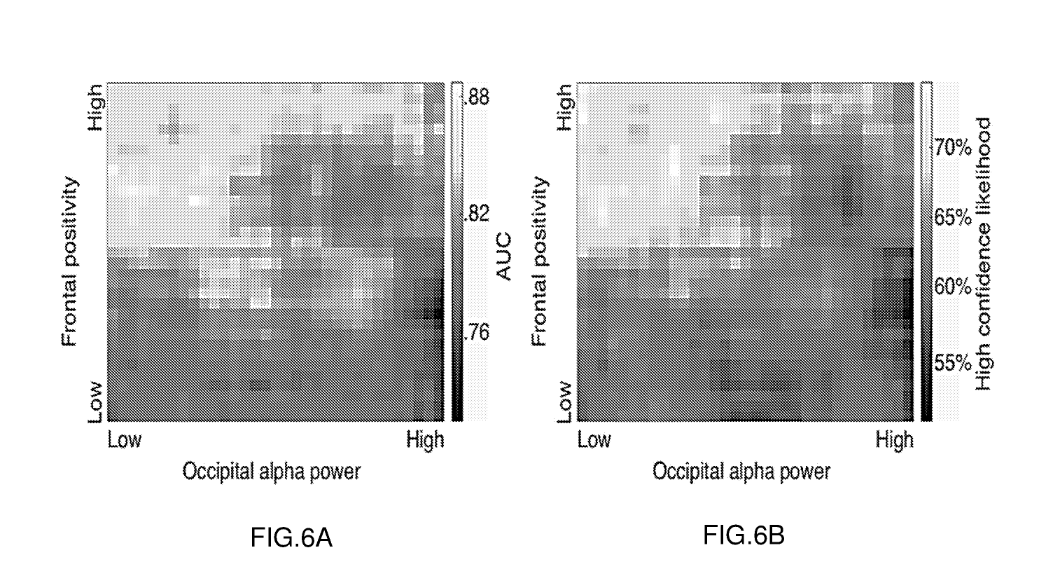

If these signals index different encoding mechanisms, then combining these measures on each trial should result in an increase in our ability to forecast later memory performance. To test this, each trial was sorted into a two-dimensional array using the frontal positivity and the occipital alpha power as two orthogonal axes. As FIGS. 6A and 6B show, for the trials with the highest frontal positivity and the lowest occipital alpha power, the AUC and the likelihood of a high-confidence response were 0.85 and 74%, respectively.

FIGS. 6A and 6B show the results of Experiment 1 in the form of heat maps depicting the combined predictive power of the frontal positivity and the occipital alpha power. In FIG. 6A, the color of each pixel indicates the area under the curve (AUC) for the corresponding values of the occipital alpha power and the frontal positivity. In FIG. 6B, the color of each pixel indicates the likelihood of a high-confidence judgment for each combination. For instance, in FIG. 6B, the very top left pixel depicts the likelihood of high-confidence responses for trials with the highest frontal positivity and the lowest occipital alpha power. As the pixel moves to the bottom, the criterion window for the frontal positivity slides lower by 2.5%. As the pixel moves to the right, the criterion window for the occipital alpha power slides higher by 2.5%.

In contrast, for the trials with the lowest frontal positivity and the highest occipital alpha power, the AUC and the likelihood of high-confidence response were 0.78 and 56%, respectively. Thus, the ability to predict later memory improved substantially when combining the two electrophysiological signals.

Experiment 2

In Experiment 2, participants studied 800 pictures while we recorded their EEG. Immediately following the initial study phase, the amplitudes of the two neural signals were used to categorize the pictures as either poorly studied or well-studied. Participants then restudied half of the poorly studied and well-studied items. If the frontal positivity and the occipital alpha power are stimulus-driven measures, then the restudy EEG signals should continue to reflect the poorly studied and well-studied categories to the same degree. However, if the two signals reflect the endogenous variance of memory encoding, then the amplitudes of restudy EEG signals should track later recognition memory performance, instead of the categories defined during the initial study phase. Additionally, if the EEG-based memory forecasting is useful in identifying objects that are poorly studied, and thus need additional studying, then one would expect that the benefit of restudying is greater for poorly studied items than for well-studied items.

Method

Stimuli and Procedures.

The initial study phase was similar to the encoding phase of Experiment 1, except that participants studied 800 pictures instead of 500. Approximately 5 min after the initial study phase, the participants completed a restudy phase, in which they restudied half of the poorly studied and half of the well-studied items, as defined by the EEG signals recorded during the initial study phase. We defined the well-studied items as those that elicited the largest 40% of all frontal positivities and the lowest 40% of occipital-alpha-power measurements. The poorly studied items were defined as those that elicited the smallest 40% of all frontal positivities and the highest 40% of occipital-alpha-power measurements. The pictures were presented in the same format as in the initial study phase. On average, participants restudied 58 poorly studied pictures and 56 well-studied pictures during restudy. After the restudy phase, participants' resting-state EEG with eyes open and eyes closed was recorded for 15 min.

Then, they performed the recognition memory test, which was identical to that in Experiment 1 except that participants were tested on five categories of pictures: poorly studied baseline pictures (58 pictures on average), well-studied baseline pictures (56 pictures on average), poorly studied restudy pictures (58 pictures on average), well-studied restudy pictures (56 pictures on average), and 160 new pictures.

Results

Participants recognized 80% of the well-studied restudy items, 80% of the poorly studied restudy items, 52% of the well-studied baseline items, and 44% of the poorly studied baseline items with 100% confidence. Of the remaining items, they recognized 9% of the well-studied restudy items, 10% of the poorly studied restudy items, 18% of the well-studied baseline items, and 21% of the poorly studied baseline items with moderate confidence (60% or 80%). Participants successfully rejected 73% of new items. Table 2 reports the proportion of trials used to derive the ROC curves. The AUC values were 0.76 for well-studied baseline items, 0.73 for poorly studied baseline items, 0.88 for well-studied restudy items, and 0.88 for poorly studied restudy items. These results demonstrate that participants learned the pictures reasonably well and benefitted from restudy.

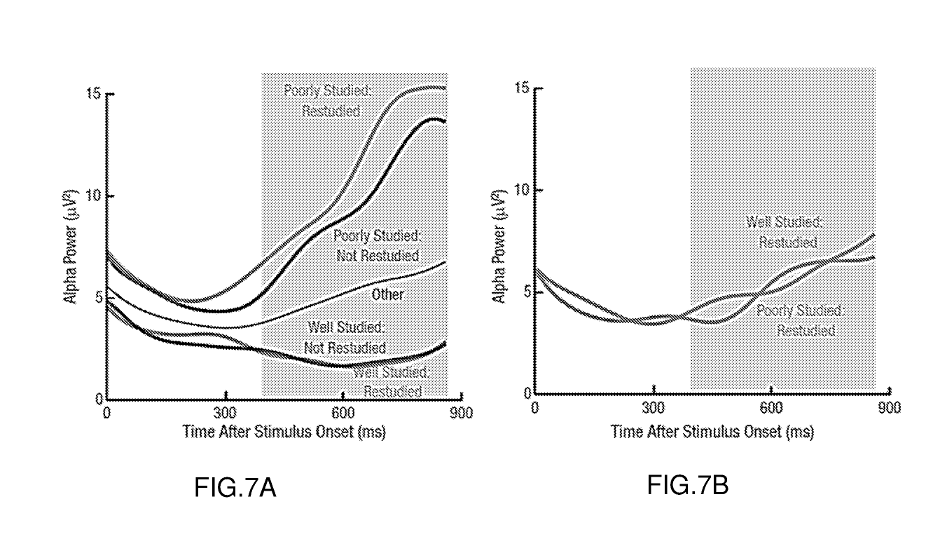

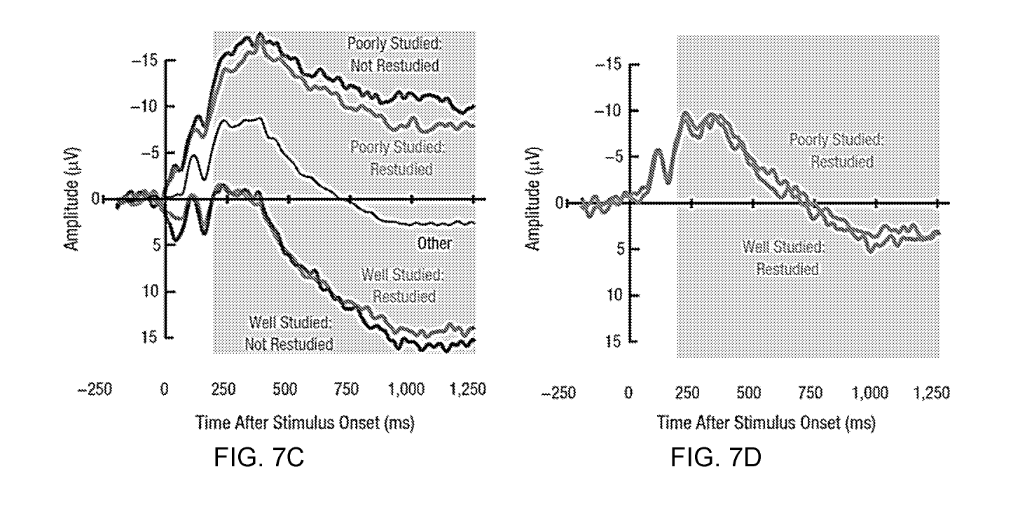

FIGS. 7A-7D shows the electroencephalogram results of Experiment 2. In particular, mean amplitude at the Fz site (FIGS. 7A and 7C) and mean occipital alpha power at the O2 site (FIGS. 7B and 7D) during the initial study phase (left column) and the restudy phase (right column). Results are shown separately for all five item types in the initial study phase and for the two restudied item types in the restudy phase. Gray shading indicates the time window over which data were averaged to quantify the amplitude of each event-related potential. The data points used to plot the curves in the bottom row represent the alpha power observed within a 400-ms sliding window that had a 20-ms step size. The values on the x-axis represent the front ends of these time windows.

In particular, FIGS. 7A-7D show the amplitude of the frontal positivity and the occipital alpha power during the initial study phase (FIGS. 7A and 7B) and during the restudy phase (FIGS. 7C and 7d) elicited by poorly studied, well-studied, and new items. The difference in the sustained frontal positivity between well-studied and poorly studied items was significant, t(19)=2.59, p<0.05 (95% CI for the difference=[0.20, 2.00 μV], Bayes factor=3.8), but much reduced during the restudy phase. The difference in the occipital alpha power was much reduced and not significant in the restudy phase, t(19)=1.57, p>0.1 (Bayes factor in favor of the null hypothesis=1.44). These findings are inconsistent with what we should have observed if the neural signals were due to the physical characteristics of the stimuli and consistent with the signals tracking the endogenous state of the participant during encoding.

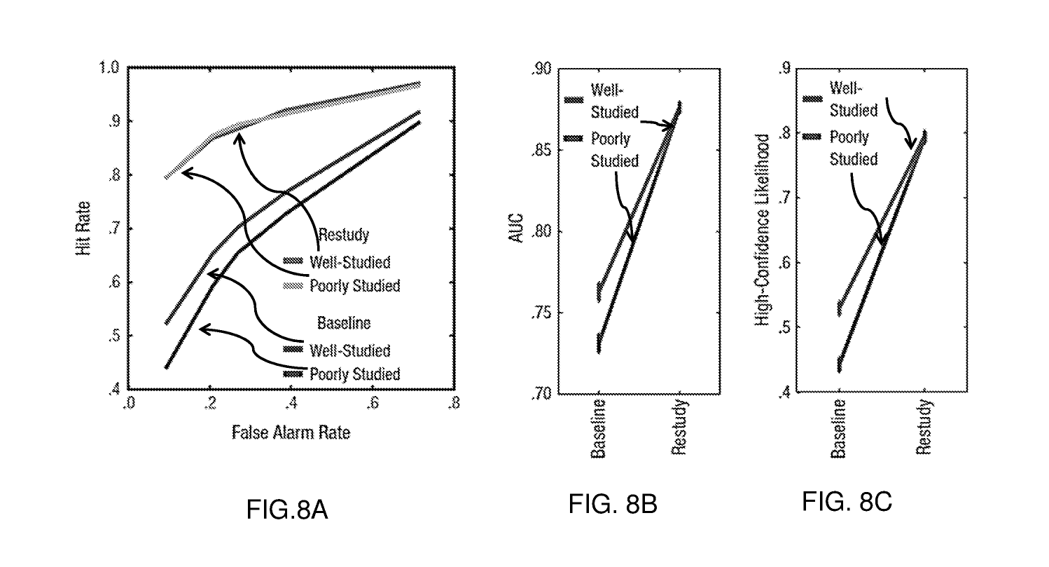

Table 2 and FIG. 8A-8C show performance from the final recognition test. FIGS. 8A-8C show the results of Experiment 2 regarding performance on the recognition memory test. The graphs show the receiver-operating-characteristic (ROC) curve (FIG. 8A), the area under the ROC curve (AUC; FIG. 8B), and the likelihood of a high-confidence response (FIG. 8C), separately for the restudy and baseline items. Error bars show standard errors of the mean.

TABLE 2
Results of Experiment 2: Mean Proportion of Responses on the Recognition Memory Test

| | Response | | | | | |
|---|---|---|---|---|---|---|
| Item type | “100% old” | “80% old” | “60% old” | “100% new” | “80% new” | “60% new” |
| Well-studied baseline | .52 (.05) | .13 (.01) | .05 (.01) | .08 (.01) | .15 (.02) | .07 (.02) |
| Poorly studied baseline | .44 (.05) | .15 (.02) | .06 (.01) | .11 (.02) | .17 (.03) | .07 (.02) |
| Well-studied restudy | .80 (.05) | .07 (.01) | .02 (.01) | .03 (.01) | .05 (.02) | .03 (.02) |
| Poorly studied restudy | .80 (.04) | .08 (.02) | .02 (.01) | .03 (.01) | .05 (.02) | .02 (.01) |
| New | .09 (.03) | .11 (.02) | .06 (.01) | .29 (.04) | .33 (.03) | .12 (.02) |

Note:
Standard errors are given in parentheses.
Proportions indicate participants' confidence in their old/new judgment.

First, the results from Experiment 1 were replicated. That is, it was found that for baseline (i.e., not restudied) items, memory strength was significantly weaker for the items that elicited a low frontal positivity and high occipital alpha power (i.e., poorly studied items) than those that elicited a high frontal positivity and low occipital alpha power (i.e., well-studied items)—for the AUC: t(19)=2.63, p<0.02 (95% CI for the difference=[0.01, 0.06 μV], Bayes factor=4.1); for the likelihood of high-confidence responses: t(19)=4.22, p<0.0001 (95% CI for the difference=[4, 13%], Bayes factor=105.0). More critically, recognition performance was essentially identical across the two types of restudied items—for the AUC: t(19)=0.24, p>0.8 (Bayes factor in favor of the null hypothesis=4.5); for the likelihood of high-confidence responses: t(19)=0.23, p>0.8 (Bayes factor in favor of the null hypothesis=4.5); there was a significant interaction between item category (poorly studied vs. well-studied) and study condition (baseline vs. restudy), F(1, 19)=8.8, p<0.01, ηₚ²=0.32, for the AUC and F(1, 19)=13.48, p<0.01, ηₚ²=0.42, for the likelihood of high-confidence responses. In fact, the restudy effect in terms of the likelihood of high-confidence responses was 1.3 times larger for poorly studied items than for well-studied items (27% vs. 35% change, respectively).

Next, the possibility was addressed that the lack of difference between recognition accuracy for well-studied and poorly studied items following restudy was simply due to a ceiling effect that eliminated the true difference that would otherwise be observed. In other words, maybe the restudy benefit was larger for poorly studied items than for the well-studied items because every restudied stimulus was relearned maximally. If so, there should be no variability left in recognition performance to be explained by the electrophysiological signatures measured during the restudy phase. To address this possibility, the restudied items were classified as poorly restudied and well-restudied on the basis of the signals recorded during the restudy phase. Again, it was found that well-restudied items had a significantly higher memory strength (M=0.92) than poorly restudied items (M=0.89) for the AUC, t(19)=2.3, p<0.05 (95% CI for the difference=[0.02, 0.52], Bayes factor=2.2) and that this was also the case for the likelihood of high-confidence responses (well-restudied items: M=0.85, poorly restudied items: M=0.78), t(19)=2.9, p=0.01 (95% CI for the difference=[2, 12%], Bayes factor=6.9). This indicated that not all the restudied items were encoded to ceiling. Instead, the variability in the encoding quality for restudied items was still distinguishable using the frontal positivity and the occipital alpha power. Therefore, the significant interaction between study condition and item category does not appear to be due to a ceiling effect for restudied items obscuring a potential difference.

Discussion

In Experiment 2, there was discrimination between exogenous and endogenous explanations of the variability in our electrophysiological indices of memory encoding. The results indicate that both the frontal positivity and the occipital alpha power heavily reflect endogenous variability in memory-encoding processes. There appears to be only a hint of exogenous contribution to the difficulty of encoding on these electrophysiological signals, evidenced by a small but preserved difference in the frontal positivity for poorly studied and well-studied items during the restudy phase. Furthermore, by having participants restudy the items that were classified as poorly studied by the electrophysiological signals, it was possible to dramatically enhance the efficacy of learning. Thus, these results provide insight as to the nature of the frontal positivity and the occipital alpha signals of memory encoding, and they provide a clear demonstration of the practicality of our EEG-based learning intervention.

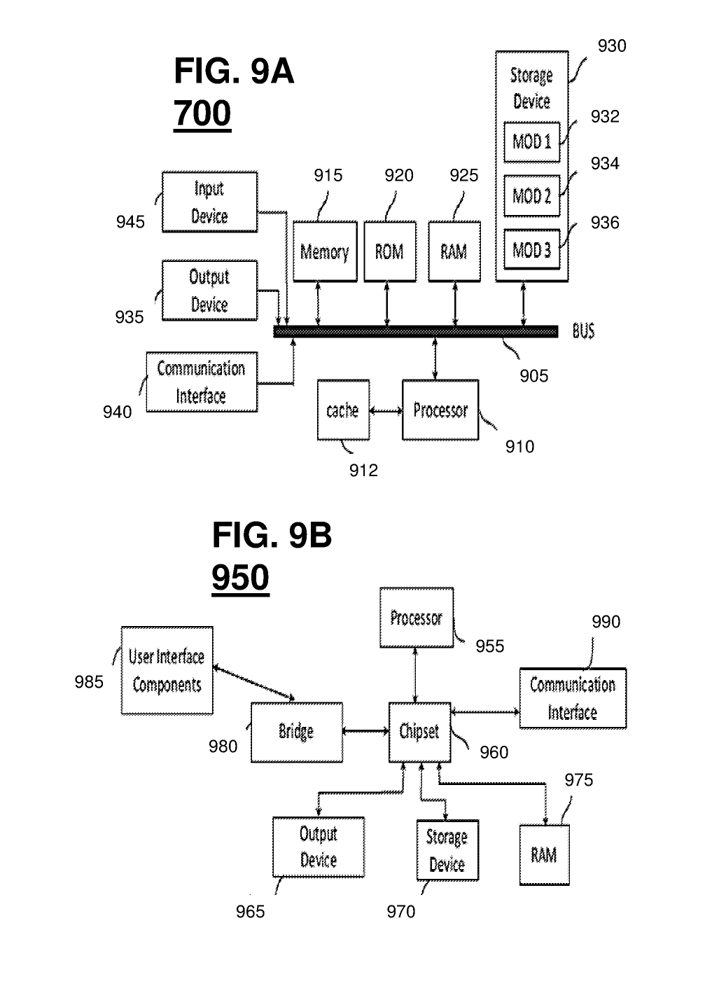

FIG. 9A, and FIG. 9B illustrate exemplary possible system configurations. The more appropriate configuration will be apparent to those of ordinary skill in the art when practicing the present technology. Persons of ordinary skill in the art will also readily appreciate that other system configurations are possible.

FIG. 9A illustrates a conventional system bus computing system architecture 900 wherein the components of the system are in electrical communication with each other using a bus 905. Exemplary system 900 includes a processing unit (CPU or processor) 910 and a system bus 905 that couples various system components including the system memory 915, such as read only memory (ROM) 920 and random access memory (RAM) 925, to the processor 910. The system 900 can include a cache of high-speed memory connected directly with, in close proximity to, or integrated as part of the processor 910. The system 900 can copy data from the memory 915 and/or the storage device 930 to the cache 912 for quick access by the processor 910. In this way, the cache can provide a performance boost that avoids processor 910 delays while waiting for data. These and other modules can control or be configured to control the processor 910 to perform various actions. Other system memory 915 may be available for use as well. The memory 915 can include multiple different types of memory with different performance characteristics. The processor 910 can include any general purpose processor and a hardware module or software module, such as module 1 932, module 2 934, and module 3 936 stored in storage device 930, configured to control the processor 910 as well as a special-purpose processor where software instructions are incorporated into the actual processor design. The processor 910 may essentially be a completely self-contained computing system, containing multiple cores or processors, a bus, memory controller, cache, etc. A multi-core processor may be symmetric or asymmetric.

To enable user interaction with the computing device 900, an input device 945 can represent any number of input mechanisms, such as a microphone for speech, a touch-sensitive screen for gesture or graphical input, keyboard, mouse, motion input, speech and so forth. An output device 935 can also be one or more of a number of output mechanisms known to those of skill in the art. In some instances, multimodal systems can enable a user to provide multiple types of input to communicate with the computing device 900. The communications interface 940 can generally govern and manage the user input and system output. There is no restriction on operating on any particular hardware arrangement and therefore the basic features here may easily be substituted for improved hardware or firmware arrangements as they are developed.

Storage device 930 is a non-volatile memory and can be a hard disk or other types of computer readable media which can store data that are accessible by a computer, such as magnetic cassettes, flash memory cards, solid state memory devices, digital versatile disks, cartridges, random access memories (RAMs) 925, read only memory (ROM) 920, and hybrids thereof.

The storage device 930 can include software modules 932, 934, 936 for controlling the processor 910. Other hardware or software modules are contemplated. The storage device 930 can be connected to the system bus 905. In one aspect, a hardware module that performs a particular function can include the software component stored in a computer-readable medium in connection with the necessary hardware components, such as the processor 910, bus 905, display 935, and so forth, to carry out the function.

FIG. 9B illustrates a computer system 950 having a chipset architecture that can be used in executing the described method and generating and displaying a graphical user interface (GUI). Computer system 950 is an example of computer hardware, software, and firmware that can be used to implement the disclosed technology. System 950 can include a processor 955, representative of any number of physically and/or logically distinct resources capable of executing software, firmware, and hardware configured to perform identified computations. Processor 955 can communicate with a chipset 960 that can control input to and output from processor 955. In this example, chipset 960 outputs information to output 965, such as a display, and can read and write information to storage device 970, which can include magnetic media, and solid state media, for example. Chipset 960 can also read data from and write data to RAM 975. A bridge 980 for interfacing with a variety of user interface components 985 can be provided for interfacing with chipset 960. Such user interface components 985 can include a keyboard, a microphone, touch detection and processing circuitry, a pointing device, such as a mouse, and so on. In general, inputs to system 950 can come from any of a variety of sources, machine generated and/or human generated.

Chipset 960 can also interface with one or more communication interfaces 990 that can have different physical interfaces. Such communication interfaces can include interfaces for wired and wireless local area networks, for broadband wireless networks, as well as personal area networks. Some applications of the methods for generating, displaying, and using the GUI disclosed herein can include receiving ordered datasets over the physical interface or be generated by the machine itself by processor 955 analyzing data stored in storage 970 or 975. Further, the machine can receive inputs from a user via user interface components 985 and execute appropriate functions, such as browsing functions by interpreting these inputs using processor 955.

It can be appreciated that exemplary systems 900 and 950 can have more than one processor 910 or be part of a group or cluster of computing devices networked together to provide greater processing capability.

For clarity of explanation, in some instances the present technology may be presented as including individual functional blocks including functional blocks comprising devices, device components, steps or routines in a method embodied in software, or combinations of hardware and software.

In some configurations the computer-readable storage devices, mediums, and memories can include a cable or wireless signal containing a bit stream and the like. However, when mentioned, non-transitory computer-readable storage media expressly exclude media such as energy, carrier signals, electromagnetic waves, and signals per se.

Methods according to the above-described examples can be implemented using computer-executable instructions that are stored or otherwise available from computer readable media. Such instructions can comprise, for example, instructions and data which cause or otherwise configure a general purpose computer, special purpose computer, or special purpose processing device to perform a certain function or group of functions. Portions of computer resources used can be accessible over a network. The computer executable instructions may be, for example, binaries, intermediate format instructions such as assembly language, firmware, or source code. Examples of computer-readable media that may be used to store instructions, information used, and/or information created during methods according to described examples include magnetic or optical disks, flash memory, USB devices provided with non-volatile memory, networked storage devices, and so on.

Devices implementing methods according to these disclosures can comprise hardware, firmware and/or software, and can take any of a variety of form factors. Typical examples of such form factors include laptops, smart phones, small form factor personal computers, personal digital assistants, and so on. Functionality described herein also can be embodied in peripherals or add-in cards. Such functionality can also be implemented on a circuit board among different chips or different processes executing in a single device, by way of further example.

The instructions, media for conveying such instructions, computing resources for executing them, and other structures for supporting such computing resources are means for providing the functions described in these disclosures.

Although a variety of examples and other information was used to explain aspects within the scope of the appended claims, no limitation of the claims should be implied based on particular features or arrangements in such examples, as one of ordinary skill would be able to use these examples to derive a wide variety of implementations. Further and although some subject matter may have been described in language specific to examples of structural features and/or method steps, it is to be understood that the subject matter defined in the appended claims is not necessarily limited to these described features or acts. For example, such functionality can be distributed differently or performed in components other than those identified herein. Rather, the described features and steps are disclosed as examples of components of systems and methods within the scope of the appended claims. Claim language reciting “at least one of” a set indicates that one member of the set or multiple members of the set satisfy the claim. Tangible computer-readable storage media, computer-readable storage devices, or computer-readable memory devices, expressly exclude media such as transitory waves, energy, carrier signals, electromagnetic waves, and signals per se.

While various embodiments of the present invention have been described above, it should be understood that they have been presented by way of example only, and not limitation. Numerous changes to the disclosed embodiments can be made in accordance with the disclosure herein without departing from the spirit or scope of the invention. Thus, the breadth and scope of the present invention should not be limited by any of the above described embodiments. Rather, the scope of the invention should be defined in accordance with the following claims and their equivalents.

Although the invention has been illustrated and described with respect to one or more implementations, equivalent alterations and modifications will occur to others skilled in the art upon the reading and understanding of this specification and the annexed drawings. In addition, while a particular feature of the invention may have been disclosed with respect to only one of several implementations, such feature may be combined with one or more other features of the other implementations as may be desired and advantageous for any given or particular application.

The terminology used herein is for the purpose of describing particular embodiments only and is not intended to be limiting of the invention. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

What is claimed is:

1. A method, comprising:
generating, during a first time period, at least one sensory stimulus for a learner;
collecting, during a second time period after the first time period, first electroencephalogram (EEG) signals for at least one first electrode site and second EEG signals for at least one second electrode site;
calculating a first characterization value based on the first EEG signals and a second characterization value based on the second EEG signals;
determining whether the first characterization value and the second characterization value fail to meet respective first and second conditions; and
in response to determining that the first characterization value and the second characterization value fail to meet the respective first and second conditions, regenerating the at least one sensory stimulus for the user.

2. The method of claim 1, wherein the first EEG signals are collected from a frontal electrode site.

3. The method of claim 2, wherein the first characterization value is based on event-related potential (ERP) signals derived the first EEG signals.

4. The method of claim 1, wherein the second EEG signals are collected from an occipital electrode site.

5. The method of claim 4, wherein the second characterization value is based on occipital alpha power signals derived from the second EEG signals.

6. The method of claim 1, wherein the at least one sensory stimulus comprises a visual sensory stimulus.

7. The method of claim 1, wherein the first characterization value and the second characterization value are one of a Z-score, a percentile value, or a voltage value.

8. The method of claim 1, wherein the first condition is that the first characterization value be less than or equal to a first threshold value, and wherein the second condition is that the second characterization value be greater than or equal to a second threshold value.

9. A system, comprising:
at least one user interface device;
an electroencephalogram (EEG) signal interface;
a processor communicatively coupled to the at least one user interface and the EEG signal interface; and
a computer-readable medium, having stored thereon a plurality of instructions for causing the processor to perform steps comprising:
generating, via the at least one user interface and during a first time period, at least one sensory stimulus for a learner;
collecting, via the EEG signal interface and during a second time period after the first time period, first electroencephalogram (EEG) signals for at least one first electrode site and second EEG signals for at least one second electrode site;
calculating a first characterization value for the first EEG signals and a second characterization value for the second EEG signals;
determining whether the first characterization value and the second characterization value fail to meet respective first and second conditions; and
in response to determining that the first characterization value and the second characterization value fail to meet the respective first and second conditions, regenerating, via the at least one user interface, the at least one sensory stimulus for the user.

10. The system of claim 9, wherein the first EEG signals are collected from an frontal electrode site.

11. The system of claim 10, wherein the first characterization value is based on event-related potential (ERP) signals derived the first EEG signals.

12. The system of claim 9, wherein the second EEG signals are collected from an occipital electrode site.

13. The system of claim 12, wherein the second characterization value is based on occipital alpha power signals derived from the second EEG signals.

14. The system of claim 9, wherein the at least one sensory stimulus comprises a sensory stimulus.

15. The system of claim 9, wherein the first characterization value and the second characterization value are one of a Z-score, a percentile value, or a voltage value.

16. The system of claim 9, wherein the first condition is that the first characterization value be less than or equal to a first threshold value, and wherein the second condition is that the second characterization value be greater than or equal to a second threshold value.

17. A non-transitory computer readable medium, having stored thereon a computer program executable by a computing device, the computer program comprising a plurality of code sections for performing steps comprising:
generating, during a first time period, at least one sensory stimulus for a learner;
collecting, during a second time period after the first time period, first electroencephalogram (EEG) signals for at least one first electrode site and second EEG signals for at least one second electrode site;
calculating a first characterization value based on the first EEG signals and a second characterization value based on the second EEG signals;
determining whether the first characterization value and the second characterization value fail to meet respective first and second conditions; and
in response to determining that the first characterization value and the second characterization value fail to meet the respective first and second conditions, regenerating the at least one sensory stimulus for the user.

18. The method of claim 17, wherein the first EEG signals are collected from an frontal electrode site, and wherein the second EEG signals are collected from an occipital electrode site.

19. The method of claim 17, wherein the first characterization value and the second characterization value are one of a Z-score, a percentile value, or a voltage value.

20. The method of claim 19, wherein the first condition is that the first characterization value be less than or equal to a first threshold value, and wherein the second condition is that the second characterization value be greater than or equal to a second threshold value.
